(19) United States
(12) Patent Application Publication
(10) Pub. No.: US 2003/0032566 A1
(43) Pub. Date: Feb. 13, 2003

(54) Mild antimicrobial liquid cleansing formulations

(75) Inventors: Emma Pawson, Cheshire (GB); Caroline Birrane, Manchester (GB); Jan Gartside, Greater Manchester (GB)

Correspondence Address: Jodi-Ann McLane, Salter & Michaelson, 321 South Main Street, Providence, RI, 02903-7128, US

(73) Assignee: Cussons (International) Limited

(21) Appl. No.: 10/159,604

(22) Filed: May 31, 2002

(30) Foreign Application Priority Data
May 31, 2001 (GB) 0113182.0

(51) Int. Cl. A61K 7/50

(52) U.S. Cl. 510/130; 510/504; 510/426; 510/512

(57) ABSTRACT

The present invention is directed to an antimicrobial liquid cleansing formulation comprising:

(1) from about 1 to about 99 wt % of a surfactant system comprising one or more surfactants;

(2) from about 0.01 to about 3.0 wt % of an acid and/or acid anhydride which buffers the pH of the composition in the range from about 3.5 to about 7;

(3) from about 0.01 to about 10 wt % of at least one phospholipid; and

(4) up to about 99 wt % of water.

The pH of the composition may also preferably be in the range from about 3.5 to about 5.5.

### DESCRIPTION

1. Technical Field

The present invention relates to a liquid cleansing composition particularly, but not exclusively, for use as a personal washing composition, for example for use in handwashing and/or bodywashing. The invention may alternatively have applications in other areas such as in dishwashing or carpet and upholstery cleansing.

2. Background of Related Art

There is consumer demand for a cleansing composition which can help to remove bacteria from the skin or other surfaces, sponges or plates.

WO 95/32705 discloses an antimicrobial liquid cleansing formulation that attempts to achieve antimicrobial effectiveness in a composition comprising a mild non-soap surfactant. This is achieved by buffering the pH of the composition to no more than 5.5, using hexanoic acid (in the examples) as the pH buffering compound. Hexanoic acid has a foul disagreeable odour and is consequently not suitable for personal care compositions. The substitution of hexanoic acid by other acids suggested in WO 95/32705 has been found not to provide good antibacterial efficacy for a broad range of bacteria. This may be because hexanoic acid (caproic acid) is used in cosmetic formulations as a surfactant/cleansing agent. It is a fatty acid and is not classed in the International Cosmetic Ingredient handbook as a pH adjuster, but as a surfactant-cleansing agent. Therefore it is arguable that the examples given in WO 95/32705 make use of the surfactant properties of hexanoic acids rather than any pH adjusting properties.

GB 2326167 discloses a rinse-off cleansing composition comprising a surfactant together with a phospholipid and an anti-bacterial agent, such as Triclosan. Such anti-bacterial agents are expensive.

### SUMMARY

An antimicrobial liquid cleansing formulation is provided comprising:

(1) from about 1 to about 99 wt % of a surfactant system comprising one or more surfactants;

(2) from about 0.01 to about 3.0 wt % of an acid and/or acid anhydride which buffers the pH of the composition in the range from about 3.5 to about 7;

(3) from about 0.01 to about 10 wt % of at least one phospholipid; and

(4) up to about 99 wt % of water.

The pH of the composition may also preferably be in the range from about 3.5 to about 5.5.

### BRIEF DESCRIPTION OF THE DRAWING

It should be understood that the drawing is provided for the purpose of illustration only and is not intended to define the limits of the disclosure. In the drawing:

FIG. 1 is a graph showing the log reduction in bacterial activity of a first sample and a second sample in relation to various bacteria.

### DETAILED DESCRIPTION OF THE ILLUSTRATIVE EMBODIMENT

The present invention seeks to provide an antimicrobial liquid cleansing formulation having superior antibacterial efficacy which does not require the presence of hexanoic acid or an anti-bacterial agent such as Triclosan.

According to the present invention there is provided an antimicrobial liquid cleansing formulation comprising:

(1) from about 1 to about 99 wt % of a surfactant system comprising one or more surfactants;

(2) from about 0.01 to about 3.0 wt % of an acid and/or acid anhydride which buffers the pH of the composition in the range from about 3.5 to about 7;

(3) from about 0.01 to about 10 wt % of at least one phospholipid; and

(4) up to about 99 wt % of water.

The pH of the composition is preferably in the range from about 3.5 to about 5.5.

The use of a synthetic phospholipid within the composition in combination with the acid provides the required antibacterial efficacy when any of a broad range of acids are used that are acceptable to the consumer.

The surfactant system may comprise any anionic, amphoteric, non-ionic, cationic or zwitterionic surfactant either alone or in combination. The surfactant may comprise at least one soap. Ideally the surfactant preferably comprises any of the following either alone or in combination: alkali metal ether sulphate, alkyl sulphate, sulphosuccinate, acyl glutamate; alkyl polyglucoside, isethionate, carboxylate, soap, alkyl sulphate, ethoxylated and non-ethoxylated metal alkyl sulphonate, sultaine, taurate, sarcosinate, sulphonate (eg alkylbenzene, olefin and secondary alkyl), ether carboxylate, glycinate, quaternary ammonium compound, polysorbate, sugar ester, alkyl phosphate, propionate, amino acid surfactant, glycinate, glyside, alkanolamides, amine oxides, ethoxylated fatty alcohols, alkyl glucamides, alkyl propionates and dipropionates and betaines.

The buffering compound or compounds ideally comprise one or more of the following:—organic acid anhydrides, organic acids and inorganic acids.

The acid buffering compound(s) preferably comprise any of the following either alone or in combination:—organic acids, preferably carboxylic acids or hydroxy carboxylic acids such as acetic acid, benzoic acid, formic acid, lactic acid, maleic acid, glycolic acid, malic acid, tartartic acid, succinic acid, fumaric acid, sorbic acid, hydroxybenzoic acid, citric acid, dehydroacetic acid, salicylic acid; or inorganic acids such as sulphuric acid, hydrochloric acid, boric acid, orthophosphoric acid or phosphoric acid.

The acid is preferably present in an amount from about 0.05 wt % to about 3.0 wt % and more preferably from about 0.05 wt % to about 1.0 wt %, although the preferred amount of acid may vary depending on the acid selected, for example about 0.1 wt % to about 0.5 wt % for citric acid, about 0.2 wt % to about 0.4 wt % for phosphoric acid and about 2.0 wt % to about 3.0 wt % for benzoic acid.

The phospholipid component preferably comprises one or more cationic synthetic phospholipid of the general formula.

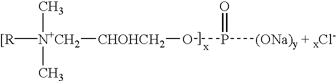

Where R=cocamidopropyl

stearamidopropyl

linoleamidopropyl

borageamidopropyl

x+y=3

The composition preferably comprises from about 0.01 wt % to about 1% wt % of the phospholipid.

The composition preferably comprises from about 60 wt % to about 90 wt % of water.

It is noted that the composition need not contain a conventional anti-bacterial agent such as Triclosan in order to achieve anti-bacterial properties. Indeed this is a significant advantage in that such anti-bacterial agents are expensive. For avoidance of doubt we have not classified preservatives or the afore mentioned phospholipid as anti-bacterial agents.

The composition may additionally comprise further minor ingredients as are commonly used in the art such as any of the following either alone or in combination:—humectant/skin-conditioning agent, skin-feel agent, preservative, chelating agent, thickener, pearliser, opacifier, viscosity adjuster, fragrance, colourant and UV absorber.

In order that the present invention may be more readily understood a specific example thereof is shown below.

A base formulation was made as set out below.

| Ingredient/INCI Name | Level (wt %) |
|---|---|
| Anionic surfactant | 7.0 |
| Amphoteric surfactant | 1.0 |
| Nonionic surfactant | 3.0 |
| Moisturizer/skin conditioner | 3.-5% |
| Preservative/Chelating agent | 0.25%-.0.35 |
| Thickeners | 0.2%-0.9 |
| Perfume | 0.70 |
| Colourant | 0.00023 |
| Aqua | Up to 100 |

A first sample of the base formulation was taken and its pH adjusted to 4.2 with a carboxylic acid (sample 1). A second sample of the base formulation was taken, phospholipid PTC was added at 0.77 wt % and the pH of the sample was adjusted to 4.2 (sample 2).

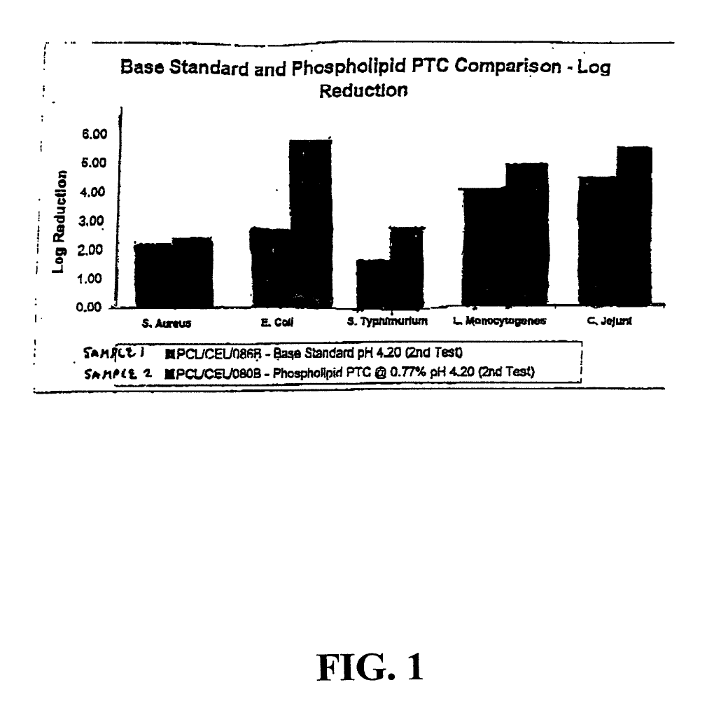

Referring to FIG. 1 attached hereto there is provided a graph showing the log reduction in bacterial activity of samples 1 and 2 in relation to various bacteria. Superior results are provided on each occasion for sample 2 as a result of adding the phospholipid to the base formulation.

It will be understood that changes in the details, quantities, materials, steps, and/or arrangement of the composition, which has been herein described and illustrated in order to explain the nature of the invention, may be made by those skilled in the art within the principle and scope of the invention as expressed in the appended claims. Therefore, the above description should not be construed as limiting, but merely as an exemplification of a preferred embodiment.

What is claimed is:

1. An antimicrobial liquid cleansing formulation comprising:
(1) from about 1 to about 99 wt % of a surfactant system comprising one or more surfactants;
(2) from about 0.01 to about 3.0 wt % of an acid and/or acid anhydride which buffers the pH of the composition in the range from about 3.5 to about 7;
(3) from about 0.01 to about 10 wt % of at least one phospholipid; and
(4) up to about 99 wt % of water.

2. The antimicrobial liquid cleansing formulation as claimed in claim 1, wherein the pH of the formulation is in the range from about 3.5 to about 5.5.

3. The antimicrobial liquid cleansing formulation as claimed in claim 1, wherein the surfactant comprises any of the following either alone or in combination: alkali metal ether sulphate, alkyl sulphate, sulphosuccinate, acyl glutamate; alkyl polyglucoside, isethionate, carboxylate, soap, alkyl sulphate, ethoxylated and non-ethoxylated metal alkyl sulphonate, sultane, taurate, sarcosinate, sulphonate, ether carboxylate, glycinate, quaternary ammonium compound, polysorbate, sugar ester, alkyl phosphate, propionate, amino acid surfactant, glycinate, glyside, alkanolamides, amine oxides, ethoxylated fatty alcohols, alkyl glucamides, alkyl propionates and dipropionates and betaine.

4. The antimicrobial liquid cleansing formulation as claimed in claim 1, wherein the acid comprises one or more carboxylic acid and/or hydroxycarboxylic acid.

5. The antimicrobial liquid cleansing formulation as claimed in claim 1, wherein the acid comprises any of the following either alone or in combination:—acetic acid, benzoic acid, formic acid, lactic acid, maleic acid, glycolic acid, malic acid, dehydroacetic acid, tartaric acid, succinic acid, fumaric acid, sorbic acid, hydroxybenzoic acid, citric acid, dehydroacetic acid, salicylic acid.

6. The antimicrobial liquid cleansing formulation as claimed in claim 1, wherein the acid comprises at least one inorganic acid.

7. The antimicrobial liquid cleansing formulation as claimed in claim 1, wherein the acid comprises any of the following either alone or in combination:—sulphuric acid, hydrochloric acid, boric acid, metaphosphoric acid or phosphoric acid.

8. The antimicrobial liquid cleansing formulation as claimed in claim 1, wherein the phospholipid comprises one or more cationic synthetic phospholipid of the general formula 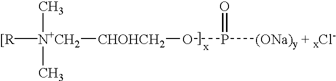
Where R=cocamidopropyl
stearamidopropyl
linoleamidopropyl
borageamidopropyl
x+y=3

9. The antimicrobial liquid cleansing formulation as claimed in claim 1, wherein the formulation comprises from about 0.01 wt % to about 1 wt % of phospholipid.

10. The antimicrobial liquid cleansing formulation as claimed in claim 1, wherein the formulation comprises from about 60 wt % to about 90 wt % of water.

11. The antimicrobial liquid cleansing formulation as claimed in claim 1, wherein the formulation comprises from about 0.05 wt % to about 1.0 wt % of the acid and/or acid anhydride.
